(19) United States
(12) Patent Application Publication
(10) Pub. No.: US 2012/0184825 A1
(43) Pub. Date: Jul. 19, 2012

(54) METHOD FOR DETECTING AND ANALYZING SLEEP-RELATED APNEA, HYPOPNEA, BODY MOVEMENTS, AND SNORING WITH NON-CONTACT DEVICE

(75) Inventor: Meir Ben David, Raa'nana (IL)

(73) Assignee: Meir Ben David, Raa'nana (IL)

(21) Appl. No.: 13/351,100

(22) Filed: Jan. 16, 2012

Related U.S. Application Data
(60) Provisional application No. 61/433,280, filed on Jan. 17, 2011.

(51) Int. Cl. A61B 5/0205; A61N 1/36; A61M 16/00; A61B 5/11; A61B 5/12

(52) U.S. Cl. 600/301; 128/204.23; 607/42

(57) ABSTRACT

A method for detecting sleep-related Apneas, Hypopneas, heart rate, body movements, and snoring events of a sleeping person. An online, adaptive detection system conditions and automatically analyzes physiological, movement-related and ambient acoustical signals to count valid snoring events, non-breathing events and calculates patient AHI (Apnea Hypopnea Index). Patient respiration, snoring, movements, presence and heart rate are continuously monitored, recorded and transmitted without requiring any sensors, electrodes, leads, cuffs, or cannulas to be attached to the patient. Additional benefits include improving the reliability of Apnea/Hypopnea detection in the patient home environment, and utilizing the method and the device for Apnea/Hypopnea and snoring positional therapy.

### REFERENCE TO RELATED APPLICATION

The present application claims benefit of U.S. Provisional Patent Application Ser. No. 61/433,280, filed Jan. 17, 2011, the disclosure of which is hereby incorporated by reference and priority of which is hereby claimed pursuant to 37 CFR 1.78(a) (4) and (5)(i).

### FIELD OF THE INVENTION

The present invention relates to medical monitoring and, more particularly, to a method and a non-contact device for continuous measurement, recording, and alert of sleep disorders; Apnea, Hypopnea and basic vital signs, for medical monitoring and positional therapy of Obstructive Sleep Apnea (OSA).

### BACKGROUND OF THE INVENTION

The National Institutes of Health (NIH) Sleep Disorders Research Plan expresses a need for methods that can monitor sleep characteristics without direct contact with the patient's body. In addition, with growing home healthcare remote monitoring, there is a growing need for an automatic method and system that more accurately and precisely detects the occurrence of sleep disorders, such as Apnea, Hypopnea, body movement and snoring while a person is sleeping in bed in his natural environment.

These goals are met by the present invention.

### Snoring

Snoring is associated with many potential health problems, including cardiovascular morbidity. Snoring also poses social problems when sleep partner is disturbed by the snoring sounds and loud snoring is often associated with limited airflow. The most common symptom of OSA is heavy and loud snoring, the most common and characteristic sign of sleep Apnea. Snorers may not realize that they have difficulty breathing at night unless there is someone (such as a bed partner) who can tell them that they snore or sound like they're holding their breath (“stop breathing” episodes), a warning that they may have OSA.

### Obstructive Sleep Apnea

Obstructive Sleep Apnea (OSA) is a common but under-diagnosed breathing disorder that occurs during sleep. It occurs when the upper airway between the back of the nose and the voice box collapses, blocking air from reaching the lungs. Because the muscles that hold our upper airway open are less active during sleep, almost everyone experiences some decrease in airflow during sleep. However, some people experience blockage in the airway during sleep, and temporarily stop breathing or breathe inadequately for repeated periods of time. When this occurs the individual is diagnosed with OSA.

OSA is diagnosed while a patient has at least 5 Apnea/Hypopnea episodes per hour of sleep.

Sleep Apnea episode is a breathing pause of at least 10 seconds and Hypopnea episode is a shallow breathing with abnormally low respiration rate, with a decrease of 50% in respiratory volume.

Hypopnea differs from Apnea; it retains some flow of air with an increase in breathing effort causing pseudo-breathing signal that presents additional challenge for non-contact monitoring.

The main OSA symptom is a daytime sleepiness.

OSA can lead to major health problems, and sufferers are known to have increased mortality rates because of increased morbidity due to cardiovascular diseases and stroke. Being prone to daytime sleepiness increases the risk of a sufferer being involved in a traffic or industrial accident. It is now recognized that at any one time about 10% of males and about 5% of females suffer from some form of sleep Apnea.

The severity of OSA is measured by the average number of Apneas and Hypopneas per hour of sleep; known as the AHI, (Apnea Hypopnea Index).

AHI 5-15 is categorized as mild OSA.

AHI 15-30 is categorized as moderate OSA.

AHI>30 is categorized as severe OSA.

For most moderate to severe OSA patients Continuous Positive Airway Pressure (CPAP) is the treatment of choice.

Other treatments include dental devices, weight loss and Positional Patient Therapy (PP).

PP is recognized for patients that have their most abnormal breathing while sleeping in supine posture (back posture).

AHI is the most significant factor that predicts the positional dependency. The prevalence of Positional Patients (PP) is higher in mild to moderate OSA patients than in severe OSA. Furthermore, because mild OSA patients are the vast majority of OSA patients, if this form of therapy is successful, it could be used by a considerable number of OSA patients.

Positional therapy is the avoidance of the supine posture during sleep for supine-related sleep Apnea patient. It is a suitable form of therapy for PP, particularly for those with a lateral AHI≦5 or ≦10.

Current Automatic Snoring and Apnea Detection Systems and their Shortcomings

A direct method currently employed for detecting snoring events is to listen to patient audio records and mark the number of such events per night. Another simple method for discriminating events is to set a fixed electronic threshold on an output of a microphone amplifier, and filter out background acoustical events which can be mistaken for snoring. Various threshold methods are currently employed, such as a threshold for the absolute value of the snoring signal; a threshold for the time-average snoring signal; a threshold for a time-derivative of the snoring signal; and so forth.

Current automatic snoring detection systems, however, are relatively inaccurate with respect to distinguishing an actual snoring event from other background noises or artifacts that may originate from or around a person during sleep.

Likewise, automatic Apnea detection systems are relatively inaccurate with respect to distinguishing an actual event from other artifacts, such as body movements that may originate from or around a person during sleep.

Placing a respiration sensor under the mattress may randomly provide an out-of-phase inspiration signal, resulting in incorrect correlation with a snoring event.

### SUMMARY OF THE INVENTION

According to the present invention, a reliable method for detecting Apnea, Hypopnea, heart rate, and snoring events in a noisy environment is achieved by refined discrimination of body motion and acoustical artifacts.

The detection of snoring and Apnea events is performed by a set of steps that progressively filter out signals unrelated to the snoring and Apnea events. In addition, the reliability of snoring detection is improved by correlating such detected events with supporting respiratory signals, assuring that phase reversal between the signals does not lead to false negatives or false positives of Apnea and Hypopnea events.

Embodiments of the present invention provide methods and apparatus for detecting the snoring and Apnea signals by hardware, software or combined hardware and software that can be correlated with additional monitoring of physiological parameters.

According to embodiments of the present invention, detection of Apnea/Hypopnea events is based on the simultaneous processing of two signals—mechanical and acoustical—that are measured with no device or instrument connected to the person body, while the subject sleeps on his bed.

The method includes capturing the mechanical signal by non-contact technology and providing data on a variety of patterns including patient breath cycles, movements and heart rate. The present invention provides a method for detecting Apnea/Hypopnea events in order to automatically calculate the AHI (Apnea Hypopnea Index) of the subject without direct contact with the patient's body and without requiring the patient to undergo laboratory tests.

### BRIEF DESCRIPTION OF THE DRAWINGS

The present invention will be more fully understood from the following detailed description of the embodiments thereof, taken together with the drawings in which:

FIG. 1 illustrates an apparatus according to an embodiment of the present invention.

FIG. 2 illustrates a method according to an embodiment of the present invention.

### DETAILED DESCRIPTION OF THE EMBODIMENTS

The principles and operation of a method and apparatus according to the present invention may be understood with reference to the drawings and the accompanying description.

### Apparatus

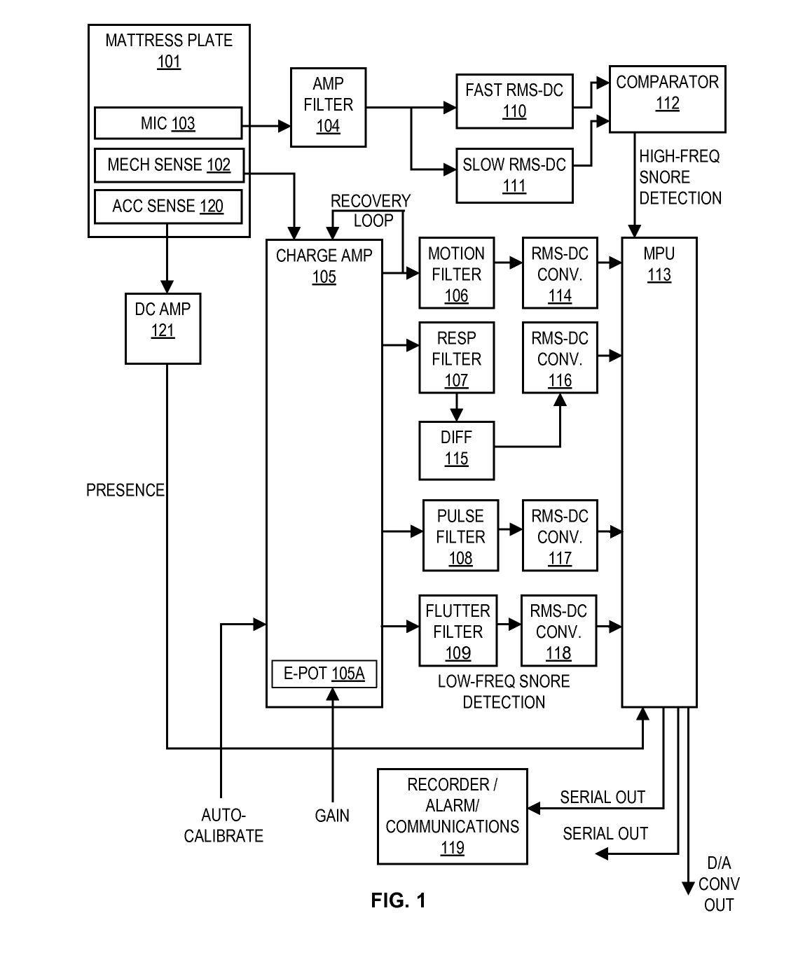

An embodiment of the present invention for use in real-time monitoring is depicted in the block diagram of FIG. 1. A plate 101 is placed under a mattress which is flexibly-supported from a frame, such as by a network of springs. In certain embodiments, plate 101 is a robust plastic incorporating grooves for flexibility and increased sensitivity to strain. Plate 101 further includes one or more of the following sensors:
A mechanical sensor 102 having an electrical output.
In certain embodiments, mechanical sensor 102 is a receiving apparatus, such as a piezoelectric disc serving as a dynamic strain gauge capable of detecting vibrations of various origins, including respiration, cardio-ballistic pulse wave, body movements, and body flutter due to sub-audible, low-frequency snoring.
A microphone 103 having an electrical output to sense audible snoring, coupled to a high-gain amplifier/filter 104.
The acoustical signal is output from microphone 103. Relatively short high intensity acoustical events correlated with breath cycles are detected as snoring. The term “snoring” herein denotes a physiological condition wherein the person's airflow is partially obstructed, creating a well-distinguished sound.
Microphone 103 listens to surrounding sounds as well as to the subject. Isolating the subject requires a contact throat microphone, which is obtrusive. A novel aspect of the present invention is the use of a contact microphone as a non-contacting microphone to sense both snoring sounds as well as stethoscopic sounds. This is implemented by using a commonly available, inexpensive contact microphone as microphone 103 and attaching microphone 103 to under-mattress plate 101. The mattress performs the acoustical coupling and amplifier/filter 104 compensates for signal loss.
In one example, microphone 103 is separate from under-mattress plate 101, and is not located under the mattress, to separate the mechanical signals from the acoustical signals.
The audible sound signal output by microphone 103 is amplified and filtered by bandpass amplifier/filter 104 to reject frequencies above the expected snoring spectrum. The signal is then converted from Root-Mean-Square (RMS) to DC by a fast responding RMS to DC circuit 110 that averages the single snoring signal, and by a slow responding RMS to DC circuit 111 that averages the ambient acoustics with the snoring signals. This filtering yields an adaptive running baseline as automatic threshold. A hardware/software comparator 112 subtracts the outputs of the two converters and creates a digital pulse representing a high frequency snoring signal. This signal is further correlated with either positive or negative derivative of the snoring signal by a differentiator 115, followed by conversion from RMS to DC by a converter 116 for input into a processor, such as a micro-processing unit (MPU) 113 which tests for an overlap of inspiration effort and snoring signal. An overlap indicates a valid snoring signal. Respiration differentiator 115 receives signals from a respiration filter 107. Finally, MPU 113 outputs a signal to a recorder/alarm/communications unit 119, by which the data output from MPU 113 (including respiration and heart rate data, and snoring, Apnea/Hypopnea detection) is recorded automatically and/or transmitted to other devices, and by which MPU 113 can signal an audible/visible alarm for patient assistance. The inputs to MPU 113 are analog-to-digital inputs. The outputs are either serial digital output, or digital-to-analog output, as noted.
A similar correlation test is performed with the low frequency, sub-audible body flutter accompanying some forms of snoring. The final decision regarding valid snoring “OR's” the two snoring signals to offer a reliable snore detector. Snoring (as well as Apnea detection) are inhibited upon sensing motion events handled by a motion filter 106 via an RMS to DC converter 114.
Microphone 103 serves another novel purpose by turning the mattress medium into a non-contact, continuous electronic stethoscope to feed a standard audio recorder (not shown).
The output comprises the physiological signals obtained by converting the AC signals to integrated DC signals. An embodiment of the present invention utilizes a bank of RMS to DC converters (114, 116, 117, and 118). RMS to DC converter 117 receives a signal from a pulse filter 108, and RMS to DC converter 118 receives a signal from a body flutter filter 109.
An acceleration sensor 120 having an electrical output and coupled to a DC amplifier 121 to sense human presence due to a body weight tilting effect, and having a presence output.
This feature reduces the probability of generating an Apnea alarm when person is not in bed. Such a sensor can also correlate respiration, cardio-ballistic pulse wave, body movements, and body flutter to enhance reliability.
Another novel aspect of an embodiment of the present invention is auto-calibration and self-test of mechanical sensor 102 and the subsequent electronic circuits could be performed. This is particularly important because sensors are subject to aging and thermal drift, for which they must be compensated. The signals processing is subject to non-adaptive thresholds, and thus the system gain must be a known quantity. Introducing periodical signals with center frequencies (respiration, pulse, motion and flutter) allows implementation of self-test and calibration. The signals are generated by MPU 113 and its internal Digital-to-Analog Converters (DAC), and the gain is adjusted automatically via electronic potentiometer (E-POT) 105A, which is part of a charge amplifier 105.

### Methods

Simultaneous analysis of mechanical signal patterns and high intensity acoustical events, in particular snoring, allows detection of Apnea and Hypopnea events.

According to embodiments of the present invention, a detection method includes the following procedures:
Detection of movement and regular patterns in a mechanical signal;
Detection of acoustical events;
Detection of Apnea events.

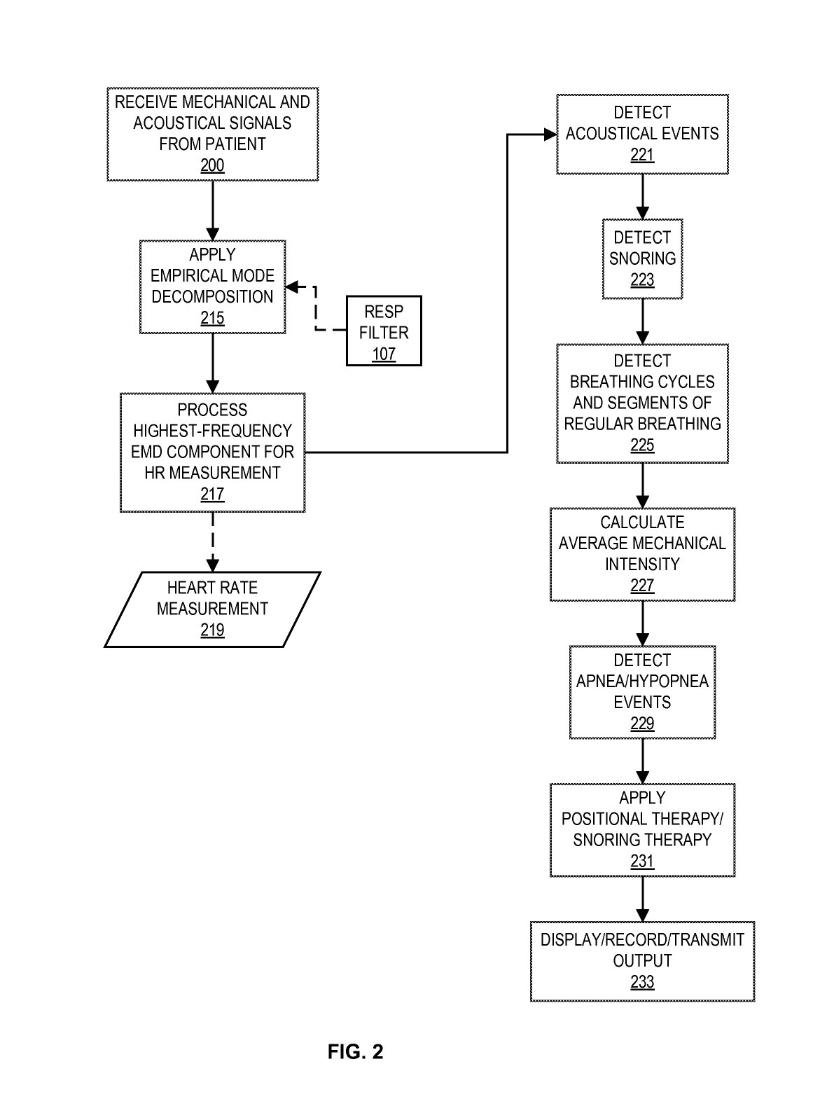

These are discussed in the following sections, with reference to FIG. 2, which illustrates a sequence of method steps according to various embodiments of the present invention.

### Detection of Movement and Regular Patterns in the Mechanical Signal

According to embodiments of the present invention, mechanical and acoustical signals are received from a patient in a step 200.

In one embodiment of the present invention, Empirical Mode Decomposition (EMD) is applied to the signal from respiration filter 107 (FIG. 1) before any other analysis.

EMD as part of the Hilbert-Huang transform, is a known technique for analyzing quasi-periodic, quasi-stationary and non-linear signals. When applied to the mechanical signal, EMD splits the signal into three components:
Fast-changing quasi-periodic pulses defined mainly by heart beats (cardio-ballistic effect);
Relatively slow-changing cycles identified with respiration and movements, which are passed to differentiator 115 for movement detection;
Residual modulations at a significantly slower rate than the breathing rate, which are ignored in certain embodiments of the present invention.

### Identification of Movement and Movement Segments

Movement detection is a requirement for any contactless technology that relies on mechanical or electro-magnetic signals for breathing/heart rate measurement. In this case, movement detection is coarse, because the goal is detecting Apnea/Hypopnea events when the subject is calm.

Intervals of mechanical signals between movement segments are referred to as non-movement intervals. According to certain embodiments of the present invention, only these intervals are examined while looking for Apnea events.

### Heart Rate Measurement

The highest-frequency EMD component—the component related to heart beats—is processed for a heart rate (HR) measurement 219 in a step 217.

In an embodiment of the present invention, HR measurement is based on calculating the power density spectrum over fixed-length windows (typically 1-3 minutes) located inside non-movement intervals.

### Detection of Acoustical Events

According to certain embodiments of the present invention, illustrated in a step 221, all intervals where the intensity of the acoustical signals exceeds the threshold as described above are considered as acoustical events. Acoustical events confined to regular breathing cycles are interpreted as snoring, which is detected in a step 223.

### Detection of Apnea/Hypopnea Events

In certain embodiments of the present invention, a specific non-movement interval is considered, and the following two preliminary steps are performed before looking for Apnea/Hypopnea events:
In a step 225 segments of regular breathing are detected.
A continuous sequence of regular breathing cycles constitutes a segment of regular breathing.
In a step 227, the average mechanical intensity for all sampling points in the current non-movement interval is calculated.
I1<I2 herein denote indices of the boundary samples (global maxima/minima) of a specific interval inside the current non-movement segment.
Consider the region bounded by a straight line S between boundary points and samples of a breath wave B between these points.
For each index n, I1<=n<=I2 calculate the following value:

AX_{K}(n)=|B(n)−S(n)|^{K}
This intensity variable may be calculated for all samples in the current non-movement segment. For each sample two values corresponding to maxima and minima of the defined intervals may be calculated:

AX_{K}(n) for maxima intervals and

AN_{K}(n) for minima intervals.
These intensities are averaged over non-overlapping equal windows that contain M samples. The value referred to as average mechanical intensity is assigned to all samples (indices) inside the averaging window.
According to embodiments of the present invention, the averaging rule is as follows:

$$∀ n ∈ Window \text{:} A ( n ) = {[ \frac{1}{2 M} {\sum }_{n ∈ Window} ( {AX}_{K} ( n ) + {AN}_{K} ( n ) ) ]}^{1 / K}$$

In an embodiment the length of the averaging window is 1 second.

After steps 225 and 227 are completed the structure of the current non-movement intervals is available. In general this includes segments of regular breathing separated by intervals where regular breathing cycles are absent.

There are two limiting cases:
The entire non-movement interval is a single segment of regular breathing;
In the current non-movement interval, regular breathing cycles are absent.

In any case the current non-movement interval is parameterized by the average mechanical intensity assigned to each point.

In the usual case when segments of regular breathing are detected, additional parameterization is related to these segments, including baseline peak-to-peak and per-breath cycle peak-to-peak.

### Specific Details for Online Implementation

In certain embodiments of the present invention, a sliding window is used for all preliminary processing.

The baseline is recalculated if:
A predefined interval has passed; or
A movement segment was detected and new breath cycles are accumulated to form the new segment of regular breathing.

To calculate the baseline update and to detect the movement segment it is necessary to obtain the minimum length of the mechanical and acoustical signals.

In addition to the sliding window containing the signal history, the history of parameters is also tracked over the history, for a length longer than the sliding window for signals.

### Apnea/Hypopnea Detection Rules

An Apnea/Hypopnea event is parameterized by a minimum duration T_{E} (subscript “E” for event) and two values of decrease in the breathing intensity: ΔP and ΔE. Apnea/Hypopnea events are detected in a step 229, according to the following rules.
ΔP is a threshold for decrease of the breath cycle peak-to-peak amplitude.
ΔE is a threshold for decrease of the average mechanical intensity.

In certain embodiments of the present invention, the following rules apply for Apnea detection.
Rule 1: For a sufficiently-long segment of regular breathing with average peak-to-peak value Pₐ, a continuous sequence of breath cycles with a duration greater than T_{E} and an average peak-to-peak decrease greater than ΔP is accepted as an Apnea event.
Rule 2. A pattern similar to that of Rule 1, but the sequence of breath cycles with low peak-to-peak value is separated from the regular segment (at one side or at both sides) by an irregular interval with an average mechanical intensity significantly higher than the same parameter at regular segments. If the irregular interval is confined to a high intensity acoustical event, then a low amplitude sequence of breath cycles is interpreted as a Hypopnea event.
Rule 3. In the case of two segments of regular breathing separated by an interval of irregular breathing whose duration exceeds T_{E}, and where the average mechanical intensity over the intermediate interval is smaller than that of two neighboring segments of regular breathing, if the decrease is less than ΔE, then the intermediate interval is interpreted as an Apnea event.
Rule 4. A pattern similar to that of Rule 4 but with a single segment of regular breathing before the irregular interval. The segment of regular breathing may occur at the end of the current non-movement interval.
Rule 5. Another pattern similar to that of Rule 4 but with an irregular interval having a sequence of subintervals with alternating small-high values of average mechanical intensity. If the high values are significantly higher than the average mechanical intensity over the regular segment and if the high mechanical intensity intervals are confined to high intensity acoustical events, then a neighboring irregular interval with sufficiently small mechanical intensity lasting longer than T_{E} is interpreted as Apnea.
Rule 6. The current non-movement interval is irregular, marked by segments where regular breathing is absent. Low/high values of mechanical intensity are compared with the most recent sufficiently-long segment of regular breathing. Corresponding thresholds may differ from similar parameters in other segments.

According to an embodiment of the present invention, in an online implementation, Apnea event detection occurs at the end of an event (e.g., movement, snoring, etc.).

### Apnea/Hypopnea Positional Therapy and Snoring Therapy

In a further embodiment of the present invention, illustrated in a step 231, the on-line output from the device and method described herein is used as a signal to control the operation of a therapeutic bed as follows:
1. Avoidance of the supine posture during sleep. Positional therapy is carried out by controlling the bed positional mechanism (head, legs, chest, or all). Detection of Apnea as disclosed herein changes the head position angle and changes the patient's posture.
2. Avoidance of the supine posture during sleep. Positional therapy is carried out by controlling the bed massage feature for several minutes of operation to change the patient's posture.
3. Avoidance of the snoring posture during sleep. Positional therapy is carried out by controlling the bed positional mechanism (head, legs, chest, or all) as disclosed herein changes the head position angle and changes the patient's posture to cause the snoring to cease.
4. Avoidance of the snoring posture during sleep. Positional therapy is carried out by activating the bed massage feature process for several minutes of operation to change the patient's posture.
5. The output from a device according to embodiments of the present invention as described herein can be used as a signal to control the operation of a second crosscheck signal, or to act as the major controlling signal for a nasal CPAP device.
6. In a further aspect of the invention, the on-line output from the device described herein is used as a signal to control an implantable sensor that provides a signal for electrical pacing of the upper airway to treat snoring or Apnea.

### Displaying, Recording, and Transmitting the Output

In another embodiment of the present invention, the on-line and off-line data and output from the method and device described herein, including respiration and heart rate data, and Apnea and snoring detection is recorded automatically and/or transmitted to other devices, in a step 233. As non-limiting examples, recording can be onto a separate memory card or other storage media, and transmitting can be via Wi-Fi or a cellular network.

The recorded or transmitted data can be delivered to professional medical staff in the relevant field directly, locally or remotely.

In still another aspect of the present invention, the on-line output can be filtered for predefined ranges or levels according to a physician's recommendation; so that an out-of-range output triggers an alarm signal that may be recorded and/or transmitted for immediate treatment and/or for further analysis by medical personnel. Non-limiting examples of such conditions include acute change in heart rate and acute change in respiratory rate.

A further embodiment of the present invention provides a computer product for performing any portion of the foregoing method of embodiments of the present invention, or variants thereof.

A computer product according to this embodiment includes a set of executable commands for performing the method on a computer, wherein the executable commands are contained within a tangible computer-readable non-transient data storage medium including, but not limited to: computer media such as magnetic media and optical media; computer memory; semiconductor memory storage; flash memory storage; data storage devices and hardware components; and the tangible non-transient storage devices of a remote computer or communications network; such that when the executable commands of the computer product are executed, the computer product causes the computer to perform the method.

While the invention has been described with respect to a limited number of embodiments, it will be appreciated that many variations, modifications and other applications of the invention may be made.

1. A method for monitoring the breathing, heart rate, motion, and sound of a resting patient and for detecting an Apnea/Hypopnea event, the method comprising:
receiving, by a receiving apparatus, a mechanical signal and an acoustical signal from the patient, wherein the mechanical signal is related to breathing of the patient and cardio-ballistic effect, wherein the receiving is performed without direct contact of the receiving apparatus with the patient;
splitting, by a processor, the mechanical signal using Empirical Mode decomposition into the following modes:
a fast-changing mode associated with heart beats; and
a slow-changing mode associated with breathing;
detecting, by the processor, a non-movement interval according to a threshold;
calculating, by the processor, an average mechanical intensity during the non-movement interval; and
detecting, by the processor, an Apnea/Hypopnea event according to a rule relating to a peak-to-peak value of the mechanical signal and the average mechanical intensity.

2. The method of claim 1, wherein calculating the average mechanical intensity comprises averaging intensities over non-overlapping equal windows.

3. The method of claim 1, further comprising setting a threshold for a decrease of a peak-to-peak amplitude.

4. The method of claim 1, further comprising setting a threshold for a decrease of the average mechanical intensity.

5. A computer product for monitoring the breathing, heart rate, motion, and sound of a resting patient and for detecting an Apnea/Hypopnea event, the product comprising a set of executable commands for performing the method according to claim 1 on a computer, wherein the executable commands are contained within a tangible computer-readable non-transient data storage medium, such that when the executable commands of the computer product are executed by the computer, the computer product causes the computer to detect the Apnea/Hypopnea event.

6. The method according to claim 1, further comprising detecting a body movement of the patient according to the mechanical signal.

7. The method of claim 1, further comprising calculating an Apnea/Hypopnea Index according to the detecting the Apnea/Hypopnea event.

8. The method of claim 1, further comprising online monitoring of the detecting the Apnea/Hypopnea event.

9. The method of claim 8, further comprising utilizing the online monitoring for Apnea/Hypopnea positional therapy.

10. The method of claim 8, further comprising utilizing the online monitoring for snoring positional therapy.

11. The method of claim 1, further comprising utilizing the detecting the Apnea/Hypopnea event to control a Continuous Positive Airway Pressure (CPAP) device.

12. The method of claim 1, further comprising utilizing the detecting the Apnea/Hypopnea event to control an implantable sensor for treating Apnea.

13. The method of claim 1, further comprising utilizing the detecting the Apnea/Hypopnea event to control an implantable sensor for treating snoring.
